(19) United States
(12) Patent Application Publication
(10) Pub. No.: US 2022/0287718 A1
(43) Pub. Date: Sep. 15, 2022

(54) OPEN-PORE SURGICAL VESSEL CLIP FOR CLOSING BLOOD VESSELS

(71) Applicant: Aesculap AG, Tuttingen (DE)

(72) Inventor: Thomas Scholten, Tuttlingen (DE)

(21) Appl. No.: 17/630,369

(22) PCT Filed: Jul. 22, 2020
(86) PCT No.: PCT/EP2020/070667

§ 371 Date: Jan. 26, 2022

(30) Foreign Application Priority Data
Jul. 31, 2019 (DE) 10 2019 120 640.5

(51) Int. Cl. A61B 17/122; A61L 31/02

(52) U.S. Cl. CPC A61B 17/122; A61L 31/022; A61B 2017/00526

(57) ABSTRACT

A surgical vessel clip, an applicator, a medical product set, and a powder metallurgical molding method for manufacturing of the vessel clip. The surgical vessel clip includes an elongate first and an elongate second retaining arm portion. The first and second retaining arm portions can be flexibly interconnected at an end of each retaining arm portion by a connecting portion. The first and second retaining arm portions include, at another end of each retaining arm portion, respective closure portions. Inner surfaces of the first and second retaining arm portions can be brought closer to each other from an open position to a closed position and can be connected to each other. At least one clip inner surface portion of the vessel clip has an open-porous design with at least one integrally formed pore, preferably with a plurality of the pores.

### CROSS-REFERENCES TO RELATED APPLICATIONS

This application is the United States national phase entry of International Application No. PCT/EP2020/070667, filed Jul. 22, 2020, and claims priority to German Application No. 10 2019 120 640.5, filed Jul. 31, 2019. The contents of International Application No. PCT/EP2020/070667 and German Application No. 10 2019 120 640.5 are incorporated by reference herein in their entireties.

### FIELD

The present disclosure relates to a surgical vessel clip, such as a ligation clip or an aneurysm clip, as used for closing blood vessels for the purpose of hemostasis. The present disclosure also relates to a method for manufacturing of the surgical vessel clip. Furthermore, a corresponding clip-applying forceps and a corresponding product set comprising the surgical vessel clip in combination with at least one accessory, such as an identification label, are proposed.

### BACKGROUND

In the prior art of open and endoscopic surgery, it is known to use surgical vessel clips or vascular clamps for closing hollow organs, such as blood vessels. On the one hand, so-called ligation clips are used for the purpose of hemostasis by means of ligation, the intended purpose of which is usually a permanent vessel closure. On the other hand, so-called aneurysm clips are used, the intended purpose of which is to permanently and/or temporarily close a vessel. Aneurysm clips of this type are marketed, for example, by the company AESCULAP AG, which markets an aneurysm clip system under the registered trademark YASARGIL® (brochure no. C45601 0810/1.0/14, which by reference is herewith expressly made a part of the present disclosure document).

First, the term hemostasis refers to all those measures that stop bleeding. In addition to the body's own or physiological hemostasis mechanisms where, on the one hand, primary or cellular hemostasis and, on the other hand, secondary or plasmatic hemostasis take place, there are various medical or surgical measures. These measures lead mechanically, thermally and/or by means of high voltage to a sealing of opened blood vessels. “Ligation” (from Latin “ligare”: “bind”), which is the sealing of blood vessels by ligating the blood vessel or tying it off, is a particularly important option among the plurality of surgical measures or techniques. The advantage of ligation is its particular reliability with which the blood vessel is securely closed mechanically. In surgery, ligation is therefore a surgical practice which is an alternative to the suturing of blood vessels and in which a hollow organ, such as a blood vessel, is ligated or tied off.

Ligation or tying-off by means of ligature frequently uses a surgical thread or, instead, a surgical ligation clip that can be applied more quickly for clamping or stapling or “clipping” a hollow organ, such as a blood vessel. Thus, WO 2007/087834 A1 of the present applicant, which by reference is herewith expressly made a part of the present disclosure document, relates to a generic vessel clip in the embodiment of a surgical ligation clip. The ligation clip disclosed therein comprises two retaining arms, which are connected to each other at a respective end by means of a deformable connection site and are bendable relative to each other in such a way that the arms move from an open position, in which they have a larger distance from each other, to a closed position, in which the mutually facing inner surfaces of the arms are permanently brought closer to each other.

In addition, it is previously known from WO 2015147440 that hemostasis by means of a vessel clip is additionally supported by a hemostatic member which suppresses bleeding and which is provided separately in the form of an inner pad. The hemostatic element surrounds the inner surfaces of the first and second legs of the vessel clip, for which purpose it is impaled on at least two pin members of the legs that protrude on the inside. To this end, the hemostatic member as a fibrous inner pad can be impregnated with a hemostatic substance, such as fibrin glue, calcium chloride, thrombin.

However, the prior art solutions involve some disadvantages. First, there is the need for the vessel clip to reliably maintain its position on the vessel in order to maintain the vessel ligation or vascular closure. However, if the surfaces of the clip inner contour are too smooth or not positionally stable, the clips can slip off the vessel because the physiological moisture of the tissue, i.e. the moisture that is based on physical and/or chemical life processes, acts like a lubricating film. Separate inner pads, as proposed in WO 2015147440, can further exacerbate the structural problem of slippage.

In addition, the real surgical situation in the case of so-called cauterization (from Latin “cauterisatio”: burning with a cautery) exhibits the additional complication that, during an ongoing operation, larger quantities of an oily lubricating body fluid mixture are constantly produced locally in the opened tissue. Here, surgical instruments for cauterization, such as an electric cauter or an electric scalpel, are used as a physical hemostasis method. To this end, a high-frequency alternating current is applied to the electric scalpel, causing immediate superficial coagulation of the blood in the cut tissue, which essentially stops the bleeding. However, a multiphase physiological (and in some cases also pathological) body fluid mixture of blood, lymph, coagulated blood cells and other components, burnt tissue or corpuscle particles and the like continuously emerges from the locally advancing cut surface.

This multiphase body fluid mixture is present as a type of oil-in-water emulsion with aqueous suspended solid fractions. This body fluid mixture with oily or lipophilic fractions reinforces the problem of the physiologically already slippery vessel surface like a lubricating layer or a physiological (or in some cases pathological) lubricating film. As a result, it becomes much more difficult for a surgeon to precisely position and clamp vessel clips in a positionally secure manner in the real-life situation of a surgical intervention.

In the case of conventional vessel clips, efforts are made to secure them against slipping by improving the static friction between the physiologically smooth, rubber-like elastic-firm vessel surface and inner contour of the vessel clip, on the one hand, by profiling the inner contour or, on the other hand, by correspondingly increasing the clip rigidity and correspondingly increasing the surface pressure. However, profiling has the disadvantage that an increased grip of the profile results in greater traumatization of the vessel surface. Profiling also generally requires additional process steps or more complex molding tools in the manufacturing process. On the other hand, increased clip rigidity leads to an increase in the load on the clip-applying forceps and to an increase in the application force, which is also to be regarded as negative from the point of view of traumatization. Further disadvantages and limitations arise from the aspects of fine-motor manual handling by the surgeon and in endoscopic interventions from the spatially and/or kinematically limited boundary conditions.

In addition, the main influencing factor causing slippage under real surgical conditions persists in the solutions of the known prior art, namely the above described circumstance of further reduced low static friction due to a lubricating layer of a multiphase body fluid mixture. This lubricating layer of multiphase body fluid mixture disadvantageously causes an inadequate surface contact between the blood vessel and the inner contour of the clip.

### SUMMARY

Thus, the object of the invention is to create a surgical vessel clip, in particular a ligation clip or an aneurysm clip, as used for closing hollow organs, such as blood vessels, for the purpose of hemostasis, which clip overcomes the disadvantages of the above described prior art. The aim is thereby to achieve good surface contact between the blood vessel and the inner contour of the clip and to largely minimize static friction in order to minimize slipping effects of the vessel clip on the vessel surface during ligating or tying-off or clamping. In particular during cauterization, e.g. with an electric scalpel, with the negative consequence of a continuous source of multiphase body fluid mixture in the opened or cauterized tissue and thus of continuous (neo)formation of lubricating layer, a surgeon should be optimally supported in his manual handling of vessel clips. An additional object is to provide a user, such as a surgeon and/or assisting clinical staff, with a corresponding clip-applying forceps and a product set that is convenient, flexible and reliably usable for today's complex clinical procedures. Furthermore, there is a need for corresponding methods for manufacturing the vessel clip that can be robustly implemented on an industrial scale in compliance with precise product specifications.

As a first aspect of the present disclosure, the surgical vessel clip used for closing hollow organs, such as blood vessels and the like, comprises: a first and a second retaining arm portion, a connecting portion, and an closure portion. Thereby, the first and second retaining arm portions are both (of) elongate (form). By means of a connecting portion of the vessel clip that is provided at a respective end of the first and second retaining arm portions, these retaining arm portions are flexibly connected to each other and/or can be flexibly connected to each other. Thereby, the first and second retaining arm portions comprise a respective closure portion at another end. In this closure portion, the mutually facing respective clip inner surfaces of the first and second retaining arm portions can be brought closer to each other, starting from an open position with a larger distance from each other, into a closed position and can be connected to each other. The closed position can be temporary or permanent, as in particular in the case of aneurysm clips the temporary or permanent closure is the intended purpose. In the case of ligation clips, the intended purpose is permanent vessel closure. For this purpose, the vessel clip is pressed into a permanent closed position, for example with the aid of a corresponding clip-applying forceps, for the ligation of the blood vessel. According to the invention, at least one clip inner surface portion of the vessel clip has an open-porous design with at least one integrally formed pore, preferably with a plurality of the pores.

The technical solution idea underlying the present disclosure is based on an increase in the frictional force by removing the physiological (or also pathological) lubricating film which is described in the introductory part and which is otherwise located between the hollow organ or blood vessel and the clip inner surface portion adjacent thereto. Therefore, a vessel clip according to the invention, which is made of open-porous material, allows the lubricating film between the clip inner contour and the blood vessel to be discharged into the at least one pore, preferably plurality of the pores. This improves the surface contact and increases the static friction between the vessel clip and the hollow organ or blood vessel. In summary, the vessel clip according to the invention has the advantage of an increased position stability on the hollow organ or blood vessel against slippage, and this without an increase in vascular trauma.

According to the invention, the surgical vessel clip for closing hollow organs comprises at least one porous surface portion in its inner contour. When this porous surface portion comes into contact or abutment with the hollow organ to be closed or clipped in the situation of surgical intervention or operation, a moist surface film present or spread on the hollow organ can be absorbed by the at least one pore, preferably by a plurality of pores. The at least partial removal of the moist surface film inherent to the hollow organ, in particular of the multiphase physiological lubricating film, from a surface of the hollow organ or blood vessel abutting the inner contour of the vessel clip into the pore(s) causes reduced or even eliminated slippage of the vessel clip on the hollow organ. In other words, the blood vessel, for example, is no longer greasy or slippery on its surface surrounded by the vessel clip while the vessel clip is being closed. Thus, instead of the vessel clip sliding or slipping on the moist surface film, essentially solid surfaces adhere or rub against one another. Therefore, the static friction between the vessel clip and the hollow organ is significantly increased during the process of closure or clipping. This makes it easier for the surgeon to apply the vessel clip securely in the correct position without having to apply traumatically high clamping forces to the hollow organ or blood vessel. The location-proof position of the vessel clip significantly improves its function and reliability. Healing is also significantly promoted due to the reduced or even eliminated local traumatization of the hollow organ or blood vessel.

The term vessel clip, as used herein, comprises a ligation clip or an aneurysm clip as described in the introductory part of the present disclosure (including the prior art).

Again, the term ligation clip also includes here so-called microclips for the purpose of temporary and/or permanent ligation of (cerebral) arteriovenous anomalies or malformations (AVM). It is understood in the sense of the present invention that in the case of microclips correspondingly smaller dimensioned scales are comprised by the subject matter of the invention. However, it must here be taken into account that, if necessary, it cannot appear to make sense to miniaturize the individual pore or an internal pore structure in its size to the same extent as the external dimensions or dimensioning of the entire vessel clip, due to manufacturing aspects and/or considerations of liquid-solid interfacial physics.

The term “integrally formed” pore or plurality of the pores is understood to mean that the pores are formed as gaps (gap volumes) or holes in the material of the vessel clip. Therefore, this term refers to a pore that is formed in a quasi materially bonded manner with regard to or in the vessel clip.

In most cases, the vessel clip forms a U- or V-shaped configuration in which the elongate retaining arm portions form the legs at the connecting portion, which is preferably arranged approximately in the center. In principle, however, the term of the type in question, i.e. vessel clip, should here not only be limited to the variant in which a vessel clip is only clamped or “clipped” at an open end opposite a connection site, i.e. it engages on one side, as is the case, for example, with the ligation clip described above in the introductory part. In the present case, the term “vessel clip” also includes any embodiments in which two-sided engagement takes place, i.e. clamping or “clipping” takes place at two ends which are initially open. Furthermore, any circumferentially closed, in particular ring-shaped or polyhedron-shaped, forms of the vessel clip are also included. It is understood that the latter closed forms can only be slipped onto a separate, e.g. cut, hollow organ or vessel, so that free portions of the vessel clip then protruding from the vessel circumference can subsequently be clamped or “clipped”.

It is also not relevant in the sense of the invention whether the different regions of the vessel clip, such as the retaining arm portions, have a specific form or a constant cross-sectional area. Thus, embodiments of vessel clips which have a variable cross-section along their developed clip length or longitudinal axis shall also be comprised herein. Cumulatively or alternatively, in addition to the two embodiments shown in the drawings, each having a rounded-rectangular cross-section, cross-sections having an oval, square, U-shaped, T-shaped, I-shaped, convex and/or concave form may be equally preferred.

Preferably, in the surgical vessel clip, the at least one open-porous clip inner surface portion is provided as a first clip inner surface portion disposed in the first retaining arm portion and/or as a second clip inner surface portion disposed in the second retaining arm portion and/or as a third clip inner surface portion disposed in the connecting portion. This offers the technical advantage that, depending on the medical indication, the overall design of the vessel clip can be optimized for its mechanical properties, such as flexural rigidity and/or degree of hardness and/or flexibility and/or elasticity. Depending on the medical history or medical indication of the respective individual case, it can be preferred, for example, that only one of the two retaining arm portions is designed with a porous clip inner surface portion, either the first or the second retaining arm portion. It can thereby be preferred that a distinction between the first and second holding arm portions, with otherwise identical external dimensions, is only determined by the temporal sequence in which the application to the hollow organ takes place during the actual surgical clipping process. This can be determined, among other things, by how or in which insertion direction the vessel clip to be applied is placed or inserted into a corresponding clip-applying forceps.

Any combination of open-porous first and/or second and/or third clip inner surface portions is included. This is because, depending on the individual nature of the vessel or body tissue to be clipped, different local designs can be advantageous. It is also conceivable that the individual or respective open-pore or porous first and/or second and/or third clip inner surface portion is arranged not only in the center but also off-center. In this context, the term “arranged” means in respective relation to the corresponding partial region of the vessel clip, i.e. in respective relation to the first retaining arm portion or to the second retaining arm portion or to the connecting portion.

Furthermore, it can be preferred that at least one partial region of the vessel clip, i.e. the first retaining arm portion and/or the second retaining arm portion and/or the connecting portion, have more than one open-pore region or porous clip inner surface portion in each case.

This leads to the additional advantage that the mechanical resistance of the vessel clip as a whole against unintentional opening can be maintained by a specific selection of the open-pore region or porous clip inner surface portion.

Preferably, the surgical vessel clip is at least 60 percent by volume, preferably at least 90 percent by volume open-pore or porous, and even more preferably (almost) completely open-pore or porous, i.e. (almost) 100 percent by volume. In this context, the restriction “almost” means that, in particular, manufacturing-related edge effects, such as non-porous injection points or geometry-related regions, such as non-porous kinks, should not be included in the corresponding determination of porosity. In a variant of this type, the vessel clip becomes particularly receptive to a greater amount of moisture or lubricating film to be removed from the hollow organ or blood vessel.

An alternative preferred variant with respect to the above mentioned preferred embodiment of the surgical vessel clip relates, precisely, to a not too high porosity or void volume fraction or gap volume portion of the vessel clip. Thereby, the vessel clip is designed to be at most 70 volume percent, preferably at most 50 volume percent, even more preferably at most 35 volume percent open-pore or porous. In particular, capillary-like and/or individual pores can be preferred. In the case of bi-continuous pore structures, e.g. in the presence of a sponge structure or a (monomodal or multimodal) sphere packing, low porosities can be preferred as they correspond to thicker wall structures, e.g. thicker framework structures, such as stronger trabeculae. Therefore, such pore structures with rather low porosity are particularly robust and mechanically strong.

Alternatively or cumulatively, the vessel clip in the alternative preferred variant comprises at least one non-open-porous clip inner surface portion, in particular with respect to the connecting portion in the clip valley of the vessel clip. Thus, the vessel clip preferred in this variant comprises at least one open-porous clip inner surface portion and at least one non-open-porous clip inner surface portion. In particular, at least the connecting portion and/or the closure portion of the first and/or second retaining arm portion is made of solid material. In a variant of this type, the vessel clip becomes particularly stable since only one or some region(s) is/are porous and/or the pore structure as such is only moderately open-porous, i.e. has some few pores per surface unit. There may also be particular manufacturing advantages, insofar as the porous design, depending on the manufacturing process and clip design, can mean increased manufacturing effort compared to solid material.

In order to achieve the desired effect on the basis of the at least one pore, it can also be sufficient and preferred to make only the inner region of the vessel clip in open-porous design, for example the inner third of the cross-sectional dimension of the retaining arm portions constituting the clip legs. Compared to a continuously open-porous cross-section, this has the advantage that the bending rigidity of the vessel clip is less reduced [symbol] A particularly advantageous vessel clip can be one the inner contour of which is not open-porous throughout but in which the region of the connecting portion representing the clip valley (in particular the proximal 20% of the developed clip length) and/or the region of the closure portions representing the clip tips (in particular the distal 10% of the developed clip length) is or are made of solid material. In particular, a design of the connecting portion representing the clip valley with solid material ensures the full bending rigidity of the vessel clip compared to a conventional vessel clip. Alternatively or cumulatively, at least a segment of an outer side clip outer surface portion of the vessel clip can be made of solid material and/or the at least one pore can be designed as a fluid drainage draining in a transverse direction, preferably bent in an L-shape toward a clip side surface, namely in that preferably the outer surface of the vessel clip has solid material and the liquid is drained laterally. This has the advantage that the porous region can be further restricted in favor of the rigidity of the vessel clip without considerably reducing the desired drainage effect of moisture or lubricating film to be removed from the hollow organ or blood vessel.

Preferably, at least one pore in the surgical vessel clip has a first pore size and/or, in the case of a plurality of the pores, an average first pore size which is 0.01 mm to 0.2 mm, preferably 0.02 mm to 0.08 mm, more preferably about 0.05 mm, at a pore entrance height at the corresponding clip inner surface portion. Alternatively or cumulatively, the (average) pore size is 1% to 35%, preferably 2% to 20%, more preferably 3% to 14%, even more preferably 5% to 10% of a clip arm width determinable in a transverse direction of a central portion of the first and/or second clip arm portion. Thus, a customary retaining arm width or web width for a ligation clip can be 0.5 to 2 mm.

The term “average pore size” in this context includes statistically formed mean values which can be averaged from density distributions according to pore diameter, pore surface area and/or pore volume. Common laboratory measurement methods for determining an (average) pore size are based, for example, on the so-called bubble point method or, for example, also on optical measurement using photo plan views and/or electron micrographs. Furthermore, (automated) measuring methods are known for determining the pore size and also the porosity, in which the individual pore or the internal pore structure is wetted by a wetting agent, such as mercury. Thereby, specific conclusions can be drawn about the variables to be measured on the basis of the capillary Laplace pressure for an individual pore as well as on the basis of the hysteresis curve.

Preferably, the at least one pore in the surgical vessel clip is capillary-like or cylindrical or singular. This has the advantage of ease of fabrication. For example, the solid material of a conventionally manufactured vessel clip can be provided with a capillary-like pore. Preferably, the at least one capillary-like pore is countersunk or drilled for this purpose with a laser or a precision drilling tool, both through-holes and blind holes being preferable.

Alternatively or cumulatively, the preferable plurality of the pores can form a bi-continuous, i.e., a continuous, pore structure with respect to one another. More specifically, the term “bi-continuous” porosity refers to a pore structure which is formed in an uninterrupted or continuous manner in two respects. Thus, both the solid material, i.e. the walls or supports, and the void volume or pore gaps are, considered separately, continuous or interconnected. This bi-continuous pore structure can further preferably be sponge-like and/or reticulated and/or fiber-knit-like and/or filamentary and/or trabecular. Furthermore, bi-continuous embodiments of the plurality of the pores with and/or without blind pores are preferably included.

The term “trabecular” or “trabecula” (from Latin trabecula ‘small beam’), as used herein, refers to a small beam-like pore structure or a small beam-like network or a network of thin webs or a sieve-like mesh. Trabecular structures are known from the internal anatomy of organs, e.g. in cardiac muscle fiber strands or in the iridocorneal angle of the eye.

A particularly preferred bi-continuous porous metal is a metal material marketed under trademark TRABECULAR METAL™ by the company ZIMMER SPINE, INC. (Edina, Minn., USA). This is a porous tantalum material used in the field of medical technology, particularly in orthopedic implantology. Trabecular metal material is a three-dimensional material with very high biocompatibility. The trabecular metal material has a porosity of up to over 80 percent by volume. Elemental tantalum is deposited onto a substrate using a thermal deposition process. This creates a sponge-like or cancellous bi-continuous pore structure in a three-dimensional material. This pore structure exhibits a uniform three-dimensional cellular architecture. The entire surface of trabecular metal material exhibits a nanostructured surface topography. In compression tests, trabecular metal material exhibits high ductility without mechanical failure, which makes it particularly advantageous for designing a vessel clip according to the invention.

This trabecular metal material with corresponding manufacturing processes is described in a plurality of patent specifications, for example U.S. Pat. Nos. 8,323,322 B2; 5,282,861; 5,443,515; and 6,063,442, the disclosures of which are herewith incorporated by reference. They describe the formation of a porous tantalum material having a trabecular pore structure on the basis of chemical vapor deposition of tantalum onto a foam-like carbon structure.

Further preferably, the vessel clip according to the invention can be made of porous metal, as is previously known for the molded bodies of the company M-PORE GmbH (Dresden, Germany). In these molded bodies, channels run through the foam, connecting the pores and thus forming a uniform, open and interconnected network of pores. In this regard, DE 102014118177 A1, the disclosure of which is incorporated by reference, teaches a method of manufacturing a foam-like metallic molded body for use as a heat exchanger on an electronic component. Thereby, the metallic molded body is printed entirely by means of 3D printing from a metallic or metal-containing raw material layer by layer in its three-dimensional form. This makes it possible to design arbitrarily shaped molded bodies on the computer and to manufacture them as a one-piece metal structure comprising solid material and foam regions. It can be provided to use a plurality of raw materials, each with a different type of metal, in the 3D printing process so that the molded body is built up from a plurality of metals in a single process step, the regions of different metal being of any shape and even interlocked. This gives the advantage that the structural properties of the metal foam, such as pore size, web width, pore shape, etc., can be specifically predetermined.

Alternatively or cumulatively, it is preferred to design the at least one pore with a pressure-stable pore structure and/or constant in comparison between the open position and the closed position. This has the additional advantage that the moisture or liquid phase or physiological lubricating film absorbed by the at least one pore from the surface of the hollow organ or blood vessel remains stored therein. In other words, the amount of fluid absorbed by the at least one pore is not forced back or squeezed out.

Preferably, the at least one pore of the surgical vessel clip has a hydrophilic and/or a pore inner surface portion that can be wetted by an aqueous fluid phase. Due to the wettability of the at least one pore at its pore entrance and/or at a further pore inner surface portion with an aqueous or hydrophilic fluid phase, there is a faster or active or spontaneous discharge of the moisture or of the multiphase lubricating film surrounding in the course of clipping with the vessel clip into the interior of the pore or into the internal pore structure as a result of wetting effects into the pore interior. In other words, active mass transport into the pore interior, namely along the wettable pore inner surface portions, takes place by wetting or spreading. In particular, with regard to the physiological lubricating film and also with regard to blood with the blood plasma, an emulsion-like and/or suspension-like multiphase with an aqueous main phase can be assumed. In this respect, even in the case of bi-continuous pore structures, the model of wetting of a fluid phase into a single capillary or cylindrical pore can be approximately taken as a basis.

Preferably, the surgical vessel clip is designed in such a way that when attaching it to the hollow organ or closing the hollow organ with it, the at least one pore, preferably the plurality of the pores, receives and/or discharges by capillary action into the pore interior and/or absorbs a surrounding fluid phase comprising components of body fluid, such as blood (corpuscles), lymph, tissue particles, body fat and/or cauterization product. This causes, in addition to the pure storage function, in the at least one pore a suction effect on the fluid phase or the active removal thereof.

Preferably, a void volume fraction and/or a gap volume fraction and/or a volume porosity of the at least one pore, preferably of the plurality of the pores, is 10% to 90%, preferably 30% to 88%, more preferably 35% to 86%, still more preferably 50% to 75%. Thereby, these relative variables are determined with respect to a clip volume portion adjacent in the clip inner surface portion. In other words, this means that it would not make sense from a metrological point of view to include adjacent regions of solid material or regions with a significantly different volume porosity in order to determine the volume porosity that can optionally be local or provided in a respective clip inner surface portion. In this respect, this means, in other words, that for the determination of the volume porosity, the volume balance envelope should coincide with a clip volume portion adjacent in the clip inner surface portion. A predefined target volume porosity is advantageous for consistent product properties as well as application properties. In particular, a well-balanced selected target volume porosity allows the mechanical properties of the vessel clip, on the one hand, and the absorption effect or storage function of the at least one pore to be controlled and adjusted to the specific application in an optimum manner.

Preferably, the interior of the one pore or the plurality of the pores or the internal pore structure of the surgical vessel clip is provided with a still fine-pored substructure. To this end, a pore inner surface portion of the at least one pore has an open-porous design with micropores of a smaller average second pore size. Thereby, the average second pore size is 1% to 20%, preferably 3% to 15%, more preferably about 10% of the first (average) pore size. Alternatively or cumulatively, the average second pore size of the micropores is 1 micrometer to 10 micrometers, preferably about 5 micrometers. The average second pore size is determinable at a micropore entrance height at the pore inner surface portion, for example, by the bubble point measurement method. Such a substructure of micropores, i.e., the second pore size, in at least segments of the pore inner surface of the larger at least one pore, i.e., the first pore size, causes a synergistic effect due to the bimodality of the pore sizes. Thus, this preferred embodiment provides the combined advantage of, on the one hand, (a) larger pore(s) for receiving, discharging and storing a larger amount of fluid phase, such as a physiological lubricating film, and, on the other hand, micropores which retain their storage effect due to physical wetting even when mechanical pressure is applied to the vessel clip.

Preferably, the vessel clip comprises the following materials or metallic, in particular powder-metallurgical, materials: metallic materials of the ISO 5832 standard for manufacturing surgical implants; titanium (e.g. Ti Grade2; Ti Grade4; Ti Grade5) or titanium alloys; tantalum or tantalum alloys; low-alloy steels for heat treatment (e.g. FN02; 100Cr6); tool steels (e.g. M2); stainless steels (e.g. stainless steels under the registered trademark NITRONIC® 50; 316L; 17-4-PH; 430; 440C); and/or other alloys (e.g. FN50; alloys under the registered trademark INCONEL® 601; Cu 99.9). In this context, the ISO 5832 standard is a series of ISO standards that define properties and test methods for forgeable, cold-formable and stainless metallic materials used for manufacturing surgical implants.

A corresponding applicator, in particular a corresponding clip-applying forceps, which is suitably set up on a vessel clip according to the invention, is proposed as a second aspect of the present disclosure. This provides the user with a perfectly shaped and designed tool. Preferably, the applicator or the clip-applying forceps can thereby be set up and/or is set up on the vessel clip according to the invention in such a way that the clamping force applied to the hollow organ in the closed position of the vessel clip does not fall below a minimum permissible first force limit. Alternatively or cumulatively, a maximum permissible second force limit is not exceeded. A product set of this type has the particular advantage that it is ensured by the manufacturer that the (force) applied by a user, such as a surgeon, when clipping or closing the hollow organ or blood vessel is sufficient to do so without pushing the fluid phase absorbed by the at least one pore out again or having a traumatizing effect on the hollow organ in a force-excessive manner. That is to say, the applicator or clip-applying forceps supports the aim that the force to be manually applied during clipping is within an optimum target half-boundary range or target range or corridor or boundary interval.

A medical product set, in particular a clip tray, for storage, transport and/or sterilization of a vessel clip according to the invention is proposed as a third aspect of the present disclosure. The medical product set or clip tray comprises a plurality of the vessel clips according to the invention. Preferably, the plurality of vessel clips according to the invention is permanently or temporarily provided or arranged in an assembly according to different sizes and/or shapes and/or degrees of hardness and/or flexural rigidities and/or materials and/or closure variants. Alternatively or cumulatively, the medical product set includes identification labels corresponding to the respective vessel clips. Such identification labels are used for documentation or information, for example for immediate reordering of the respectively used clips. Alternatively or cumulatively, an applicator corresponding to the vessel clip according to the invention, in particular a clip-applying forceps, is included. In particular, a product set of this type supports a targeted surgical preparation. Furthermore, there are advantages with regard to clinical procedures which concern aspects of storage, transport and/or sterilization.

As a fourth aspect of the present disclosure, a powder-metallurgical molding method for manufacturing of a vessel clip according to the invention is proposed. In this regard, the powder-metallurgical molding method of a vessel clip according to the invention as a component or product comprises sequential steps as follows: Providing a powder-metallurgical feed material or a so-called feedstock, e.g. by mixing and/or kneading and/or extruding; molding the feed material to form a near-net-shape fine-grained and/or coarse-grained green body in the shape of the vessel clip, optionally oversized according to a debinding and/or sintering shrinkage; optional debinding the green body to form a brown body; and sintering the green body, optionally debound to the brown body, to form the finished vessel clip.

In the first step of providing a powder metallurgical feed material, a homogeneous feed material or feedstock is provided. Preferably, the feed material is a metal-binder mixture comprising at least one organic binder in addition to a fine metal powder. It may be preferable, from case to case, to obtain the metal-binder mixture pre-assembled and/or to mix it previously to the mold-forming step. Previous mixing can preferably be carried out in a mixer or kneader that can be operated batchwise and/or continuously and/or by means of screw-type extrusion.

The subsequent step of shaping the feed material to form the green body can thereby take place in a molding tool, preferably by means of powder injection molding and/or powder pressing. Alternatively and/or cumulatively, the step of molding the feed material to form the green body can be carried out in an additive manufacturing step, preferably by means of metal 3D printing. Alternatively and/or cumulatively, the step of molding the feed material to form the green body can be carried out in a continuous manufacturing step, preferably by means of extrusion, even more preferably by means of filament extrusion. Optionally, the finished vessel clip can then be subjected to a further post-treatment, such as a surface finish, in a subsequent step.

Where applicable, any binder optionally present in the feed material is removed from the green body or preform of the vessel clip in a subsequent debinding step again so that a debound green body or a brown body is obtained: If a thermally removable binder is used for the feed material, debinding is carried out in a debinding oven. A thermally removable binder on the basis of polyolefin-wax blends can thereby be used. If at least part of the binder can be extracted by organic solvents, i.e. a (partially) soluble binder is present, debinding is carried out alternatively and/or cumulatively to thermal debinding using solvent extraction. Thus, it is preferred in the present case to use partially soluble binder systems in the feed material, in which, for example, a polyethylene-based polymer component is mixed with a wax. Alternatively and/or cumulatively, it can be preferred to use a catalytically degradable binder system in the feed material. With regard to the latter variant, a binder system on the basis of the catalytic degradation of polyoxymethylene (POM) by strong acids is preferably used, for example a binder system marked under the trademark CATAMOLD™ from BASF. A major advantage of the above mentioned binders or binder systems is the high green strength of the molded vessel clips.

In the subsequent sintering step, the, optionally debound, green body is sintered at high temperature in a sintering furnace. The sintering furnace can thereby be different from the debinding furnace or, in the case of a continuously operated plant, can be arranged in a further downstream furnace stage or annealing section. Preferably, the sintering can also be carried out as a laser sintering process.

The green body or brown body is compacted or sintered by sintering to form a finished vessel clip according to the invention with its final properties in terms of geometry, mechanical behavior, internal material structure and/or porosity/porosities, apart from the influences of a further possible post-treatment step. The result is a vessel clip that behaves inertly in the patient's body because it is purely metallic.

In an optional step, a further post-treatment of the vessel clip is carried out. Preferably, this is a surface finish and/or a coating, preferably of the pore inner surface portion. Preferably, the wettability of the at least one pore with a fluid phase to be absorbed by it can be specifically adjusted by means of a specific coating, for example a hydrophilic-lipophilic-balance-functional (HLB-functional) coating.

Alternatively or cumulatively, it is conceivable that the fabrication of an open-pore region of the vessel clip according to the invention is realized by applying a porous coating or a coating forming the at least one pore to solid material.

Preferably, (metal) powder injection molding represents in the present case a preferred variant of the powder-metallurgical molding method of a vessel clip according to the invention as a component or product. Powder injection molding (briefly the PIM method) or metal injection molding (briefly the MIM method) is a casting or primary molding method for the manufacturing of metallic or also ceramic components of complex two- or three-dimensional geometry. Powder injection molding is a further development in materials science and variant of the injection molding technology for thermoplastics. Particularly because of the three-dimensionally complex geometry of the vessel clip, with its pronounced curves and angles, powder injection molding offers particular advantages in terms of simpler molding compared with machining and/or forming manufacturing methods.

As a preferred variant of the powder metallurgical molding method, (metal) powder injection molding combines the mechanical advantages of sintered components with the great molding versatility of injection molding. In addition to the great freedom of design, further advantages of powder injection molding include functional integration; the elimination of numerous post-processing steps in the sense of undercuts, cross holes, blind holes, threads, surface structures, reproduction of logos, etc.; a flexible selection of materials; and cost-effective series production.

In this context, the preferred powder injection molding or PIM method or MIM method comprises successive steps: providing a powder-metallurgical PIM/MIM material as a feed material or so-called feedstock, e.g. by mixing and/or kneading and/or extruding; injection molding the feed material as molding into a molding tool to form a near-net-shape fine-grained and/or coarse-grained green body in vessel clip shape, preferably oversized according to a debinding and/or sintering shrinkage; debinding the green body to form a brown body; and sintering the brown body or debound green body to form the finished vessel clip.

In the powder injection molding step as a preferred embodiment of the molding step, a green body or preform of the vessel clip is manufactured as an injection molded part, for which purpose the feed material is injected into a tool mold having a corresponding hollow geometry or cavity. In this process, the feed material is preferably injected in liquefied form, more preferably at elevated temperature, into the closed tool mold. Thereby, the powder injection molding can be controlled by a specific temperature control so that the feed material ideally first completely fills the mold and plasticizes only afterwards. The resulting green body or preform is already close to contour or already exhibits essential external geometric features of the finished vessel clip.

Preferably, powder injection molding is carried out at injection pressures above the ambient pressure, preferably greater than 60 bar, and even more preferably greater than 90 bar. It is understood that the injection pressures to be provided on the machine side can increase even further in the case of complex and/or miniaturized geometries of the vessel clips. Insofar as particularly constricted flow cross-sections and/or extended flow paths are present, the preferred injection pressures increase in accordance with the disproportionately occurring flow resistances along the inner walls of the molding tool corresponding to the geometry of the vessel clip.

Preferably, in addition to powder injection molding, (metal) powder pressing represents in the present case a further preferred variant of the powder metallurgical molding method of manufacturing a vessel clip according to the invention. In the powder pressing step as a preferred embodiment of the molding step, the feed material, namely metal powder without or with binder, is provided to a die tool mold and compacted by means of a corresponding male tool mold under high pressures of a machine press to form a green body or preform of the vessel clip. As in the case of powder injection molding, powder pressing is also a particularly cost-effective manufacturing method which can uniformly meet the high quality requirements of medical products.

Preferably, a powder metallurgical feed material is provided in which the filler content of the feed material relative to the metal powder is less than 75 percent by volume, preferably less than 60 percent by volume, still more preferably less than 45 percent by volume. The filler content determines both the internal pore structure and the shrinkage size of the finished sintered vessel clip. A higher filler content thereby leads to lower sintering shrinkage or a lower gap volume fraction of the finished vessel clip. Furthermore, if the filler content is too high, the feed material can no longer be processed in the molding step, such as injection molding in particular, because its viscosity and/or abrasiveness is then too high.

Preferably, in the powder metallurgical molding method, the molding step for forming the green body is carried out in a tool mold, preferably by means of powder injection molding and/or powder pressing. Alternatively or cumulatively, the molding is carried out in an additive manufacturing step, preferably by means of metal 3D printing. Alternatively or cumulatively, the molding is carried out in a continuous manufacturing step, preferably by means of extrusion, still more preferably by means of filament extrusion. These methods have the advantage of large-scale usability with the high quality assurance demands of medical engineering products.

In summary, the vessel clip according to the invention with at least one pore has the advantage of increased positional stability of the clip on the hollow organ or blood vessel against slipping off without increasing the vascular trauma. The present invention thus relates to a vessel clip which is open-pore throughout or has open-porous portions. As a result, liquid films on tissue surfaces, such as the hollow organ or blood vessel to be closed, are selectively removed so that the vessel clip according to the invention is better secured against slippage. Therefore, the vessel clip according to the invention exhibits increased positional stability on the blood vessel to be ligated. A further advantage of a preferred embodiment of the vessel clip according to the invention is that the resistance of the vessel clip against opening by selective choice of the open-pore region is maintained.

Finally, it should be noted that the vessel clip according to the invention is not limited to be used solely for closing blood vessels for the purpose of hemostasis. The invention is equally advantageous for similar medical indications or uses, particularly for the variety of surgical situations and procedures. For example, a lymphatic vessel can be closed by a vessel clip according to the invention. In particular, the surgical treatment of both humans and animals is based on the same basic principles and objectives.

In this respect, the present terms, i.e. surgical vessel clip, such as a ligation clip or an aneurysm clip, comprise all specifically useful designs and/or size dimensions and/or materials. For example, in the field of use of microclips, one has to assume a comparatively smaller scale of the corresponding vessel clip according to the invention. For example, this can be the case in the field of hand and/or microsurgery. In particular, for the surgical treatment of children, especially of newborns, a modified size ratio of pore size to absolute clip size can be used. Thereby, as a consequence of different reduction requirements for different aspects of the vessel clip geometry, relations of pore size to other vessel clip dimensions can be shifted. Accordingly, an embodiment adapted or dimensioned in scale to the size of the hollow organ or blood vessel or body tissue to be clipped while maintaining the technical effects, in particular one miniaturized to the present ratio factor, cannot be excluded but is comprised by the present disclosure.

### BRIEF DESCRIPTION OF THE DRAWINGS

FIG. 1 is a perspective front view according to a first exemplary embodiment of a vessel clip according to the present disclosure in open position;

FIG. 2 is a longitudinal view according to the first exemplary embodiment;

FIG. 3 is a top view according to the first exemplary embodiment of the vessel clip in open position;

FIG. 4 is a width view (transverse view) according to the first exemplary embodiment of the vessel clip in open position;

FIG. 5 is a cross-section along line A-A of FIG. 3 according to the first exemplary embodiment of the vessel clip in open position;

FIG. 6 is a perspective front view according to a second exemplary embodiment of a vessel clip according to the present disclosure in open position;

FIG. 7 is a longitudinal view according to the second exemplary embodiment;

FIG. 8 is a top view according to the second exemplary embodiment of the vessel clip in open position;

FIG. 9 is a width view (transverse view) according to the second exemplary embodiment of the vessel clip in open position;

FIG. 10 is a cross-section along line A-A of FIG. 8 according to the second exemplary embodiment of the vessel clip in open position;

FIG. 11 is a microscopic cross-section according to a third exemplary embodiment of the vessel clip with a trabecular bi-continuous pore structure;

FIG. 12 is a schematic flow diagram of the powder metallurgical molding method of manufacturing a vessel clip according to the present disclosure.

### DETAILED DESCRIPTION

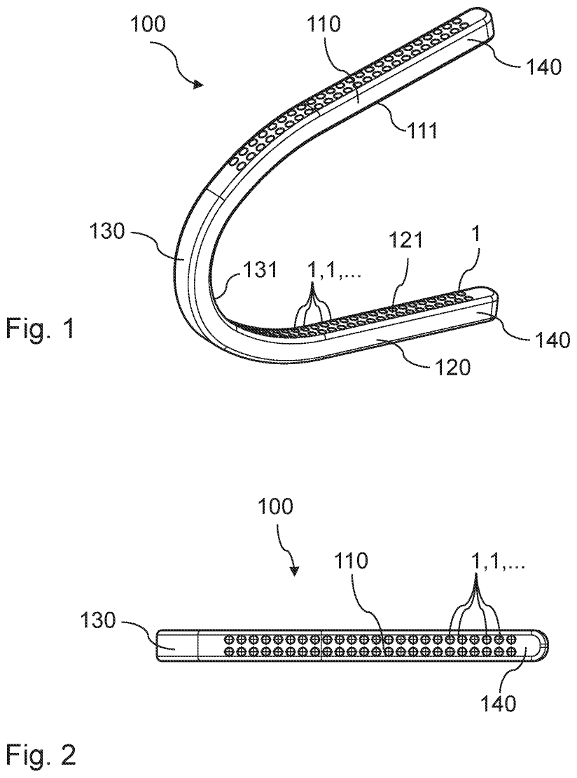

A first exemplary embodiment of the present disclosure is described below on the basis of the accompanying FIGS. 1 to 5. From this, further details, features and advantages of the invention will be apparent.

FIGS. 1 to 5 show different views according to a first exemplary embodiment of a ligation clip 100 according to the invention as a vessel clip for closing blood vessels. In the first exemplary embodiment, the ligation clip 100 is open-porous in segments each with a plurality of cylindrical capillaries 1, 1, . . . as through-holes.

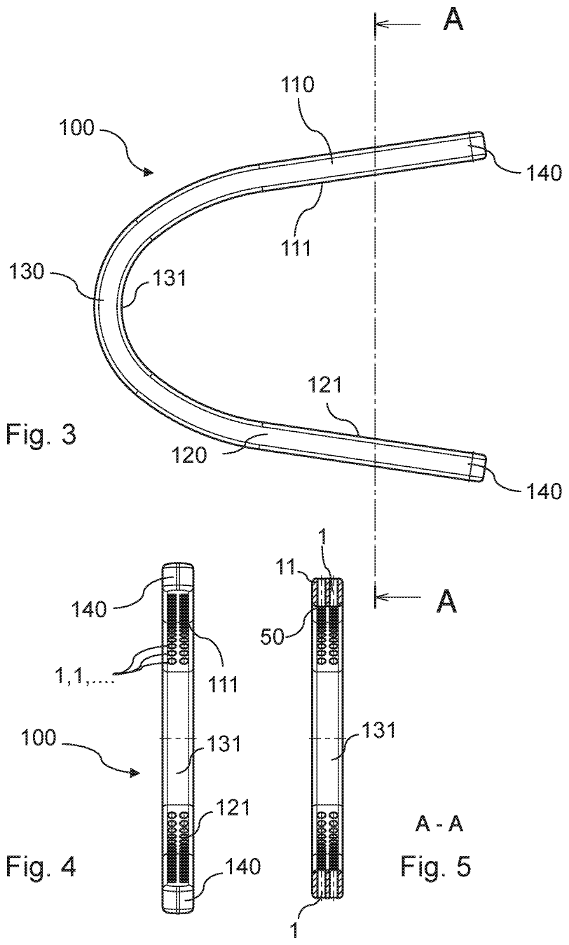

FIG. 1 shows a perspective front view of the ligation clip 100, which is bent in a U-shape and is in the open position. Two elongate clip webs 110, 120, which are of the same shape and are spaced apart from one another in an approximately parallel or slightly angled manner, as the first and second retaining arm portions open at their end located on the left in FIG. 1 into a U-shaped rounded clip valley 130 as the connecting portion of the ligation clip 100. The two clip webs 110, 120 are flexibly connected to each other via the half-arch-shaped, bendable clip valley 130 so that, starting from the open position shown in FIG. 1 or FIG. 3, where they have a larger distance from each other, they can be brought closer to each other into a closed position. For example, this is done (not shown) in that a surgeon as a user first places the U-shaped open ligation clip 100 by means of a corresponding ligation clip-applying forceps in the correct position around the blood vessel to be closed adjacent thereto and then compresses it to tie off or ligate the blood vessel. For secure permanent ligation, the two clip webs 110, 120 have a respective closure portion 140, 140 on their other end opposite the clip valley. In this way, the mutually facing respective clip inner surfaces of both clip webs 110, 120 can be permanently connected to each other.

If one mentally developed (not shown) the ligation clip 100 with the constant, slightly rounded quasi-square cross-sectional area (see FIGS. 4 and 5) along its entire clip length into an elongate square bar shape, the clip portions to be distinguished would smoothly merge into one another as follows: closure portion 140 located on the right top of FIG. 1, first clip web 110 (FIG. 1, top), half-arch-shaped clip valley 130, second clip web 120 (FIG. 1, bottom), closure portion 140 located at the right bottom in FIG. 1.

Along the inner contour of the U-shaped bent ligation clip 100, inner side clip inner surface portions should be associated with the above mentioned clip portions 140, 110, 130, 120, 140 to be distinguished. Thereby, as is clear from FIGS. 1, 2, 4 and 5, the two clip webs 110 and 120 each have in their respectively corresponding clip inner surface portions 111 and 121, respectively, a row of two with a plurality of cylindrical capillaries 1, 1, . . . as a plurality of pores, namely two times 24 capillaries per clip web 110 or 120 (thus a total number of 96 capillaries in the ligation clip 100).

In contrast, the bendable clip valley 131 is made of solid material without any capillary 1, as is clear from FIGS. 1, 2, 4 and 5.

As is clear from the cross-sectional view of FIG. 5 (cross-section along line A-A of FIG. 3), the individual capillaries 1, 1, . . . are each designed as cylindrical through-holes. The capillaries 1, 1, each have a first pore diameter as a first pore size with which they enter the clip inner surface portions 111 or 121 at a respective pore entrance height 50 on the inside, relative to the ligation clip 100. Insofar as they are cylindrical through-holes, the capillaries or capillary-shaped pores 1, 1, . . . of this first embodiment of the ligation clip 100 emerge equally at the outside clip outer surface portions with a constant pore diameter, which is an optional feature, but not any structural one essential to the invention. The retaining arm width or web width can be determined in a cross-section with, for example, a similar position as shown in FIG. 5, for which purpose the cross-section should be selected perpendicular to a longitudinal axis of one of the clip webs 110, 120.

FIG. 4 further shows the cylindrical pore wall jacket as pore inner surface 11, which encloses the cylindrical gap volume or void volume of the respective capillary 1. The latter void volume is used (not shown) in the surgical application to accommodate the physiological (or pathological) lubricating film of the blood vessel to be ligated surrounding the open-porous clip inner surface portion 111 or 121.

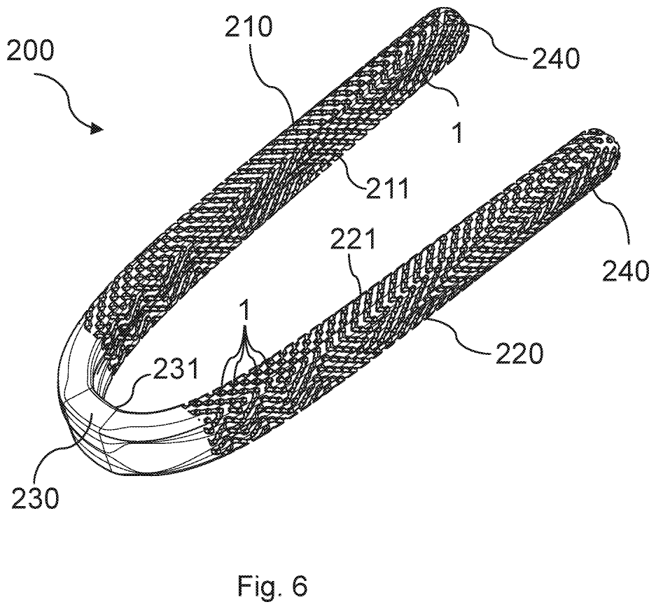

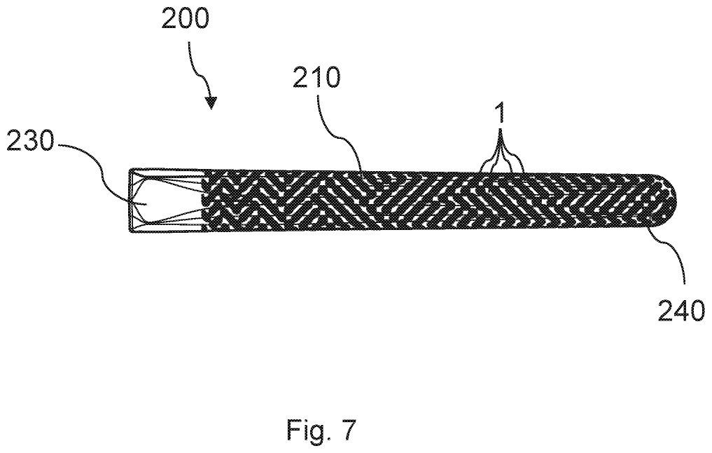

FIGS. 6 to 10 illustrate the design details according to a second embodiment of a ligation clip 200 according to the invention as a vessel clip for closing blood vessels. In this connection, the different views of FIGS. 6 to 10 according to the second exemplary embodiment show representations analogous to the views of FIGS. 1 to 5 according to the first embodiment. In order to avoid repetitions, reference is therefore made with regard to the basic structure of the ligation clip 200 (with rectangular cross-section), which is likewise bent in a U-shape, to the explanations on FIGS. 6 to 10.

In the second exemplary embodiment, the ligation clip 200, in contrast to the first exemplary embodiment of the ligation clip 100, is carried out in open-pore fashion not with capillary-like pores but with a transversely permeable or continuous, namely bi-continuous pore structure 1, 1, . . . on the basis of a plurality of pores. Due to the sponge-like pore structure, non-round shapes can be seen at pore entrance height 50.

The porosity of the ligation clip 200 according to the second exemplary embodiment is significantly higher compared to that of the ligation clip 100 according to the first exemplary embodiment, which can be geometrically derived from the bi-continuously open pore structure in the case of a comparable pore entrance size.

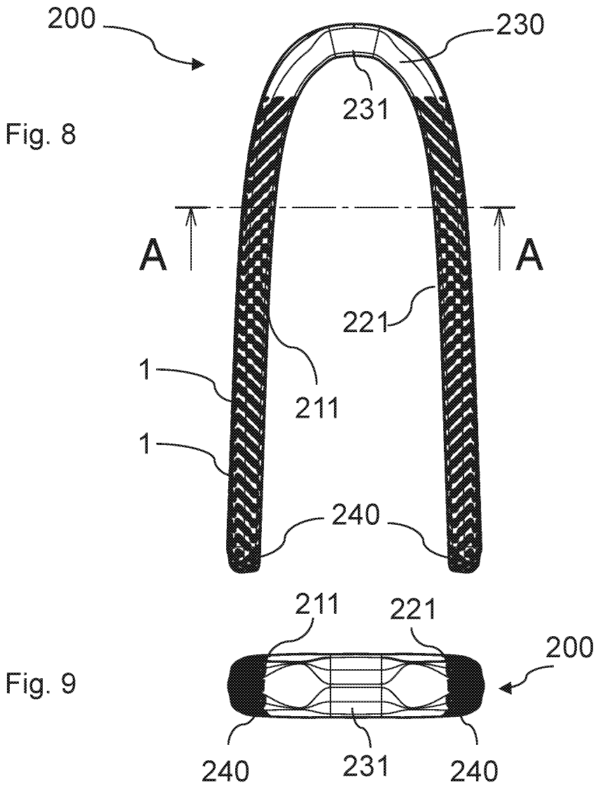

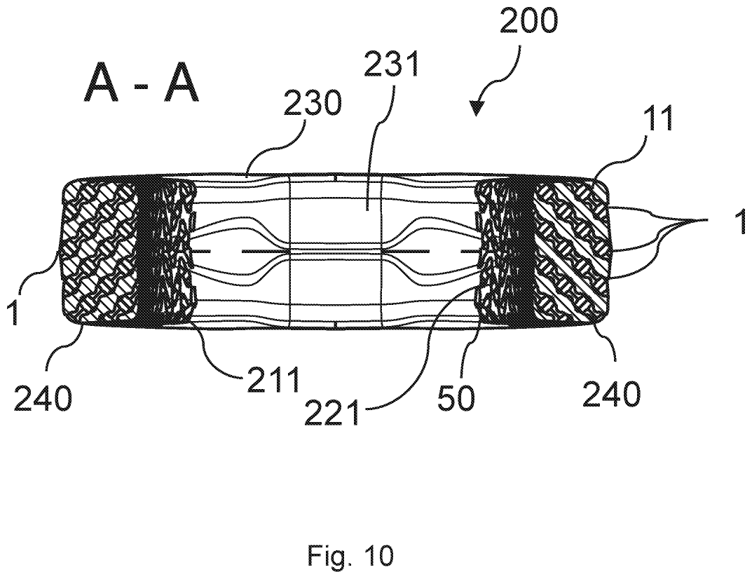

In an alternative or cumulative variant (not shown) of the first or second exemplary embodiment of the vessel clip, at least a segment of an outside clip outer surface portion of the vessel clip can be made of solid material, namely in that preferably the outer side of the vessel clip comprises solid material and the liquid is drained laterally. This has the advantage that the porous region can be further restricted in favor of the rigidity of the vessel clip without significantly reducing the desired drainage effect of moisture or lubricating film to be removed from the hollow organ or blood vessel. In particular, the at least one pore can be configured to drain fluid in a transverse direction, preferably bent in an L-shape towards a clip side surface. In the first exemplary embodiment according to FIGS. 1 to 5, this presupposes pores 1, 1, . . . of the ligation clip 100 configured as bores, which would be recognizable in FIG. 5 as an L-shaped connection (not shown) of the first or second clip inner surface portions 111, 112 with the clip side surface. In the second exemplary embodiment according to FIGS. 6 to 10, an outwardly facing partial volume, preferably one third, of the cross-section of the ligation clip 200 could be solid material (not shown) in the cross-section according to FIG. 10, along the line A-A of FIG. 8.

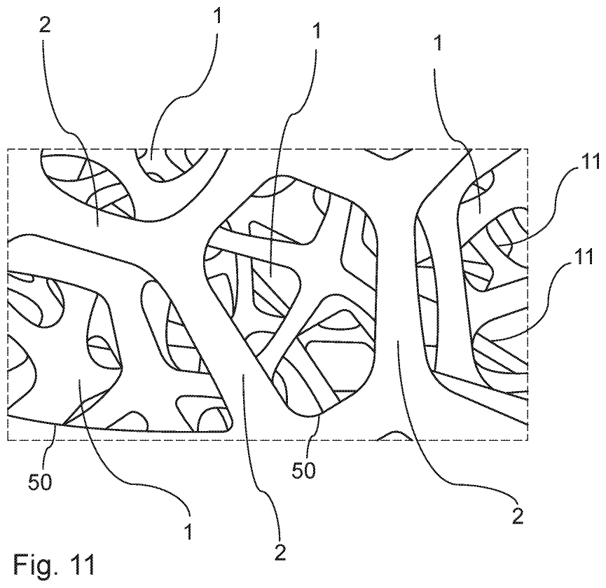

FIG. 11 shows a microscopic cross-section according to a third exemplary embodiment of the vessel clip with a bi-continuous pore structure, which is designed in a trabecular shape. A continuous mesh or open network of webs or trabeculae 2 forms a highly open-porous, bi-continuous pore structure. For example, the center of FIG. 11 shows how five trabeculae 2 form a pore 1 at pore entrance height 50 in the manner of an oblique pentagon. Further, it can be seen that the trabecular pore walls have respective pore inner surfaces 11. The entire internal pore surface area as the accessible internal solid surface area results as the totality of the individual trabecular surfaces, e.g. experimentally determinable via differential adhesion measurements, etc.

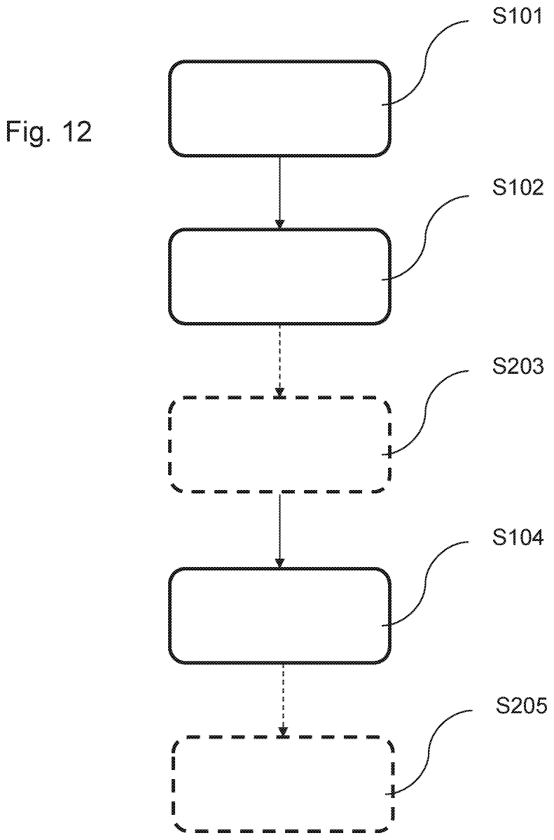

The method for the powder metallurgical molding of a vessel clip, schematized in FIG. 12 with the flow chart, comprises steps S101, S102 and S104 that are essential to the invention (shown as boxes outlined with a solid line) and further optional steps S203 and S205 (shown as boxes outlined with a dashed line): In a first step S101, a powder metallurgical feed material is provided. The feed is carried out, for example, by mixing in a mixer and/or by kneading in a kneader and/or by extruding in an extruder, preferably continuously or quasi-continuously. In a subsequent step S102, the feed material is molded to form a near-net-shape green body in the form of the vessel clip. The granularity or grain size distribution of the green body can thereby be adjusted to fine and/or coarse grains. In this way, the desired porosity in the finished product of the vessel clip can be specifically influenced. Preferably, three-dimensional oversizing is thereby carried out according to a sintering shrinkage which is preferably determined beforehand. In an optional step S203, green body is debound to form a brown body, while oversizing, if necessary, is carried out thereby on the basis of debinding shrinkage that is preferably determined beforehand. In a subsequent step S104, the molded body, i.e. the green body, or possibly the brown body debound in the optional debinding step S203, is sintered to form the finished vessel clip. Preferably, a post-treatment of the sintered vessel clip can still take place in an optional step S205. Preferably, this is a surface finish and/or a coating, in particular of the pore inner surface portion.

1.-15. (canceled)

16. A surgical vessel clip for closing hollow organs, the surgical vessel clip comprising:
a first retaining arm portion and a second retaining arm portion, each of the first and second retaining arm portions being elongate and having:
a connecting portion by which the first and second retaining arm portions have one end flexibly interconnected; and
at their other end a respective first and second closure portion with a, respectively, first clip inner surface and second clip inner surface mutually facing each other by which the first and second retaining arm portions are connectable to each other,
at least one clip inner surface portion of the surgical vessel clip being open-porous with at least one pore having a first pore size of 0.01 mm to 0.2 mm and being integrally formed, and
at least one of the connecting portion, the first closure portion or the second closure portion is made of solid material.

17. The surgical vessel clip according to claim 16, wherein the at least one clip inner surface portion is provided as one or more of:
a first clip inner surface portion arranged in the first retaining arm portion; and
a second clip inner surface portion arranged in the second retaining arm portion.

18. The surgical vessel clip according to claim 16, wherein the surgical vessel clip is at least 60 percent by volume open-pore or porous.

19. The surgical vessel clip according to claim 16, wherein the surgical vessel clip is at most 70 percent by volume open-pore or porous.

20. The surgical vessel clip according to claim 16, wherein at least a segment of an outside clip outer surface portion of the surgical vessel clip is made of solid material.

21. The surgical vessel clip according to claim 16, wherein the at least one pore is a fluid drainage in a transverse direction, being bent in an L-shape toward a clip side surface.

22. The surgical vessel clip according to claim 16, wherein the first pore size is 0.02 mm to 0.08 mm.

23. The surgical vessel clip according to claim 16, wherein the first pore size is 1% to 35% of a retaining arm width determinable in a transverse direction of a central portion of the first retaining arm portion and/or second retaining arm portion.

24. The surgical vessel clip according to claim 16, wherein the at least one pore comprises a plurality of pores, and wherein an average pore size of the plurality of pores is 0.02 mm to 0.08 mm.

25. The surgical vessel clip according to claim 16, wherein the at least one pore comprises a plurality of pores, and wherein an average pore size of the plurality of pores is 1% to 35% of a retaining arm width determinable in a transverse direction of a central portion of the first and/or second retaining arm portion.

26. The surgical vessel clip according to claim 16, wherein the at least one pore is capillary-like.

27. The surgical vessel clip according to claim 16, wherein the at least one pore is a plurality of pores that form a bi-continuous pore structure.

28. The surgical vessel clip according to claim 16, wherein the at least one pore has a pressure-stable pore structure.

29. The surgical vessel clip according to claim 16, wherein the at least one pore includes a hydrophilic pore inner surface portion.

30. The surgical vessel clip according to claim 16, wherein, relative to a clip volume fraction adjacent in the at least one clip inner surface portion, a void volume fraction and/or a volume porosity of the at least one pore is 10% to 90%.

31. The surgical vessel clip according to claim 16, wherein a pore inner surface portion of the at least one pore is open-porous with micropores of a second pore size, which at a micropore entrance height at the pore inner surface portion is 1% to 20% of the first pore size.

32. The surgical vessel clip according to claim 16, further comprising the following metallic materials:
powder metallurgical materials; and/or
metallic materials of the ISO 5832 standard for the manufacture of surgical implants; and/or
titanium or titanium alloys; and/or
tantalum or tantalum alloys; and/or
low alloy steels for heat treatment, tool steels, stainless steels; and/or
other alloys.

33. A surgical vessel clip for closing hollow organs, the surgical vessel clip comprising:
a first retaining arm portion and a second retaining arm portion, each of the first and second retaining arm portions being elongate and having:
a connecting portion by which the first and second retaining arm portions have one end flexibly interconnected; and
at their other end a respective first and second closure portion with a, respectively, first and second clip inner surface mutually facing each other by which the first and second retaining arm portions are connectable to each other,
at least one clip inner surface portion of the vessel clip is open-porous with a plurality of pores being integrally formed;
at least one of the connecting portion, the first closure portion or the second closure portion is made of solid material; and
the plurality of the pores form a bi-continuous pore structure.

34. The surgical vessel clip according to claim 33, wherein the bi-continuous pore structure is sponge-like and/or reticulated and/or fiber-knit-like and/or filamentary and/or trabecular and/or comprises blind pores.

35. A surgical vessel clip for closing hollow organs, the surgical vessel clip comprising:
a first retaining arm portion and a second retaining arm portion, each of the first and second retaining arm portions being elongate and having:
a connecting portion by which the first and second retaining arm portions have one end flexibly interconnected; and
at their other end a respective first and second closure portion with a, respectively, first and second clip inner surface mutually facing each other by which the first and second retaining arm portions are connectable to each other,
at least one clip inner surface portion of the vessel clip is open-porous with at least one pore being integrally formed,
at least one of the connecting portion, the first closure portion or the second closure portion is made of solid material, and
the at least one pore has a pressure-stable pore structure.
